(19) United States
(12) Patent Application Publication
(10) Pub. No.: US 2016/0282343 A1
(43) Pub. Date: Sep. 29, 2016

(54) QUANTITATIVE LATERAL FLOW ASSAY STRIPS FOR QUANTITATIVE ANALYSIS OF AN ANALYTE, KITS CONTAINING SUCH STRIPS AND METHODS OF MANUFACTURE AND USE OF SAME

(71) Applicant: Immunolab LLC, Lisle, IL (US)

(72) Inventors: Rajasingam S. Jeyendran, Lisle, IL (US); Seth Levrant, Oak Park, IL (US); Eugene Pergament, Chicago, IL (US)

(73) Assignee: Immunolab LLC, Lisle, IL (US)

(21) Appl. No.: 13/968,304

(22) Filed: Aug. 15, 2013

Related U.S. Application Data
(60) Provisional application No. 61/683,666, filed on Aug. 15, 2012.

(51) Int. Cl. G01N 33/543; G01N 33/573; G01N 33/74

(52) U.S. Cl. CPC G01N 33/54386; G01N 33/743; G01N 33/573; G01N 2333/928

(57) ABSTRACT

The invention is directed to quantitative lateral flow assay strips for a quantitative analysis of specific analyte by a point of care scanner. The quantitative lateral flow assay strip includes a body portion dimensioned for operable interface with the optical reader portion of the point of care scanner. The strip further including a sample pad for deposit of a known quantity of a biological sample containing an unknown quantity of the specific analyte and a conjugate pad located adjacent to the sample pad. The conjugate pad having an analyte/antibody conjugated to an optically detectable particle, which are all fixed to the conjugate pad in a dry state. The strip also has a capillary flow portion adjacent to the conjugate pad having a pore size selected to facilitate sample flow through the body portion and a test line portion having an anti-analyte antibody fixed in a dry state to the body portion of the strip. The strip may also include a control line having anti-conjugate antibody fixed in a dry state. In response to the presence of the specific analyte, the test line displays an optical intensity which is proportional to the quantity of analyte in the sample. The invention further comprises a kit including quantitative lateral flow assays strips, as well as method of manufacture and use of such strips.

### FIELD OF THE INVENTION

The present invention is directed to test strips, methods and kits for conducting quantitative lateral flow analysis of an analyte in a biological sample. More particularly, the invention is directed to such systems, methods and kits that make it possible to accurately quantify the amount of analyte in a biological sample at either a point of care facility, in the field away from a laboratory or in third world hospital settings by analysis of a quantitative lateral flow assay strip for a particular analyte of interest with an inexpensive, point of care optical analyzer.

### BACKGROUND OF THE INVENTION

Currently, quantitative analysis of an analyte in biological sample required the services of a specialized laboratory. This is because immunoassays that provide a quantitative measurement of the amount of an analyte (or analyte concentration) in a sample have previously used complex, multistep procedures and expensive analyzers available only in a laboratory setting Immunochromatographic assays such as described in U.S. Pat. Nos. 5,096,837; 5,238,652 and 5,266,497 provide simple comparative values for analytes, but do not provide quantitative measurements of analyte. Instead, these immunochromatographic assays detect the presence or absence of an analyte above a defined minimum cutoff level for the test being performed. They do not provide either a definite concentration or amount of the analyte in the sample.

There are currently a variety of lateral flow systems for detecting the presence of an analyte in a biological specimen. U.S. Pat. No. 6,136,610 (the '610 patent) illustrates one approach to detecting the presence of analyte in a sample. The '610 patent discloses the use of a dedicated reader and test strip to provide increased reliability to lateral flow assays to test for the presence or absence of a specific analyte. The '610 patent discloses ranking analyte sample test bands into low (1), medium (2), and high (3) optical density valves relative to the optical density of the control band, but does not disclose quantifying an actual amount or concentration of an analyte in a biological specimen.

In-vitro fertilization embryo transfers (“IVFET”) process in which an egg is fertilized by the sperm outside her body and the resultant embryo is transferred back into her body. IVFET is a major treatment for infertility especially when other methods of treatment have failed. In order to increase the chance of fertilization, patients are given ovulation induction medications, which coax the ovaries to produce more than one egg to maturity during each fertility cycle. This process increases serum hormone levels of estrogen (estradiol) in the patient to much higher than normal values. Analysis of estradiol levels is critical to achieving appropriate follicle development during IVFET procedures. When serum levels of estradiol is inadequate, follicular development is less than desired and the treatment often fails because insufficient numbers of eggs can be gathered to a have a good chance of achieving IVFET. If levels of this hormones become too high, ovarian hyper stimulation syndrome (OHSS) may occur which, in the best case scenario, can lead to multiple fetuses being conceived in vitro. In the worst cases, OHSS can lead to serious complications such as, excessive fluid retention in the patient's abdomen and/or chest cavity; thrombosis of arteries and/or veins (formation of blood clots) which may lead to stroke, embolus, or potentially fatal complications; or abnormally enlarged ovaries, which have the possibility of rupturing or twisting (a surgical emergency). These serious complications can result in prolonged hospitalization and, in rare cases, can even be fatal.

For these reasons, IVFET patients currently have blood samples drawn at a physician's office or clinic on a daily basis or as needed by the physician during ovarian stimulation protocol each fertility cycle. The blood samples are transported by a licensed medical courier to an off-site licensed, diagnostic laboratory for analysis of serum estradiol levels. The laboratory typically conducts either a radio immunoassay (RIA) or an enzyme immunoassay (ELISA) to determine the estradiol concentration of the patient's blood. Both of these quantitative testing procedures typically take several hours and must be performed by trained laboratory personnel using specialized, expensive equipment in a laboratory setting. Consequently, the monitoring of IVFET patient hormone level is quite expensive. The results of the estradiol quantitative analysis are typically communicated to the patient via telephone call by the physician's office. Frequently, the patient's current medication dosage must be modified in response to the estradiol assay results. In some cases, especially in the third world countries, this will require the patient to, once again, travel back to the physician's office for injection or intravenous application of additional medications all in the same day as the initial blood draw. Further, since the patient typically has to undergo the same venipuncture, testing process early the next morning and the prolonged waiting process, during each fertility cycle, most patients find the process to be tedious, expensive and overly time-consuming. For these reasons, applicant believes there is generally a need for a more convenient, cost effective method for quantifying an analyte in biological samples, and in particular, there is a need for a quick, convenient, cost effective system and method for quantifying estradiol blood concentrations for IVEFT patients.

### OBJECTS OF THE INVENTION

Accordingly, it is one object of the invention to provide test strips for real-time quantitative analysis of an analyte at a point of care facility.

It is another object of the invention to provide a kit containing such test strips and all other consumable materials necessary for real-time, cost efficient, quantitative analysis of an analyte in a biological sample at a location remote from a laboratory.

It is another object of the invention to provide a method for real-time, cost efficient, quantitative analysis of an analyte within a point of care facility thereby greatly reducing or eliminating the delay and costs associated with sending biological samples to off-site laboratory facilities for quantitative analysis.

It is still another object of the invention to provide a series of quantitative lateral flow test strips for different analytes with each different strip providing for efficient, low-cost, quantitative analysis of a specific analyte.

It is a further object of the invention to provide quantitative lateral flow test strips designed for quantitative analysis of specific analytes by inexpensive, simple to operate, handheld or desk top optical scanning devices which can be operated by healthcare facility staff, thereby greatly reducing or eliminating the need for highly trained laboratory staff and expensive equipment for quantitative analysis of the specific analytes.

It is a still further object of the invention to provide test strips for cost efficient, real-time quantitative analysis of serum estradiol concentrations at a point of care facility by health facility staff thereby greatly reducing or eliminating the need for highly trained laboratory staff and expensive equipment for quantitative analysis of serum estradiol levels during IVFET procedures.

It is also an object of the invention to provide a test strip having sufficient sensitivity that patient's estradiol levels or levels of similar biological analytes can be determined from saliva samples provided by IVEFT patients or other patients in a point of care setting.

It is a still further object of the invention to provide test strips for cost efficient, real-time quantitative analysis of serum estradiol concentrations which may eliminate the need for a trained Phlebotomist to daily draw blood from IVFET patients in order to quantify serum estradiol levels.

### SUMMARY OF THE INVENTION

In one aspect of the invention, a quantitative lateral flow test strip is provided which is calibrated for a specific analyte and designed for quantitative analysis by a point of care scanner having a strip receiver portion. The quantitative lateral flow assay strip includes an elongated body portion dimensioned for insertion into the strip receiver portion of the point of care scanner; a sample pad for deposit of a known quantity of a biological sample containing an unknown quantity of the specific analyte; a conjugate pad located adjacent to the sample pad and having an analyte/antibody conjugated to an optically detectable particle, which are fixed to the conjugate pad in a dry state; a capillary flow portion adjacent to the conjugate pad having a pore size selected to facilitate sample flow through the elongated body portion; a test line portion having an anti-analyte antibody fixed in a dry state to the body portion of the strip; and a control line having anti-conjugate antibody fixed in a dry state; in response to the presence of the specific analyte, the test line displays an optical intensity which is proportional to the concentration (amount) of analyte in the sample. The point of care scanner may be either an optical scanner which measures the visible light either absorbed or transmitted at the test line (either directly or through image analysis) or may be a fluorescent scanner which measures florescent light emitted at test line. Accordingly, for purposes of this application, the phrases “analyzing the optical intensity” and “optical intensity readings” of the assay strip test line shall refer to either the quantification of light transmitted or absorbed at the test line whether from a fluorescent optically detectable particle (Immunofluorescent stain) or a colored, conventional detectable particle (conventional immunological stain). Preferably, the point of care scanner is pre-programmed to interpret the optical intensity reading for the quantitative lateral flow test strip, calculates a concentration (amount) of analyte, and displays the calculated concentration value to an operator in real time at the point of care facility. It is also preferred that the elongated body portion is further dimensioned to be inserted into a cassette housing which is itself dimensioned for receipt in the strip receiver portion of the point of care scanner. Optionally, the scanner may be linked to the point of care facilities computer system so that the rest results may be stored electronically in the memory of that system in a specified patient's medical records. Also, optionally, the quantitative lateral flow assay strip may include a barrier portion to prevent cells and non-cellular particulates that may be found in the biological sample from entering the remainder of the quantitative lateral flow assay strip. The quantitative lateral flow assay strips may be designed for both competitive assay techniques and for sandwich assay techniques. The sensitivity of the strips of the invention make it possible to provide antibody based quantitative analyte assays for relatively small blood volume samples on the order of between about 0.05 ml to about 0.1 ml. Such small blood volume samples can typically be drawn via a finger prick method, instead of traditional venipuncture techniques. Due to their sensitivity, the strips of the invention may also be used to quantify analyte samples in a patient's saliva thereby, in some cases, completely eliminating the necessity of drawing any blood to quantify an analyte associated with a medical condition.

In another aspect of the invention, a quantitative lateral flow assay kit is provided, which contains the consumables constituents necessary for quantitative analysis of a specific analyte in a point of care operated scanner. The kit includes the following: (1) a chase buffer selected to optimize sample lateral flow through the test strip and to chemically stabilize the analyte during the assay procedure and (2) a quantitative lateral flow assay strip having (a) an elongated body portion dimensioned for insertion into a strip receiver portion of the point of care scanner; (b) a sample pad for deposit of a known quantity of a biological sample containing an unknown quantity of the specific analyte; (c) a conjugate pad located adjacent to the sample pad and having an analyte/antibody conjugated to an optically detectable particle, which are fixed to the conjugate pad in a dry state; (d) a capillary flow portion adjacent to the conjugate pad having a pore size selected to facilitate sample flow through the elongated body portion; (e) a test line portion having an anti-analyte antibody fixed in a dry state to the body portion of the strip, the test line displaying variable optical intensity proportional to the concentration (amount) of analyte in the sample; and (f) a control line having anti-conjugate antibody fixed in a dry state, the control line displaying a relatively stable optical intensity in the presence of the analyte. Preferably, the scanner is pre-programmed to interpret the optical intensity reading for the quantitative lateral flow test strip, to calculate a concentration (amount) of analyte, and to display the calculated concentration valve to an operator in real time at the point of care facility. Optionally, the scanner may be linked to the point of care facilities computer system so that the test results may be stored electronically in the memory of that system in a specified patient's medical records. Also, optionally, the quantitative lateral flow assay strip of the kit may include a barrier portion to prevent cells and non-cellular particulates that may be found in the biological sample from entering the remainder of the quantitative lateral flow assay strip. The quantitative lateral flow assay strips of the kit may be designed for both sandwich assay and competitive assay techniques. In the former assay, optical intensity is directly proportional to analyte concentration, and in the latter assay, optical intensity is inversely proportional to analyte concentration.

### BRIEF DESCRIPTION OF THE DRAWINGS

The organization and manner of the structure and function of the invention, together with the further objects and advantages thereof, may be understood by reference to the following description taken in connection with the accompanying drawings, and in which:

FIG. 1 is a schematic view of a quantitative lateral flow assay strip in accordance with one preferred embodiment of the invention;

FIG. 2 is a schematic view of a sandwich assay embodiment of a quantitative lateral flow assay strip of the invention illustrating the initial condition of the strip upon deposition of biological sample;

FIG. 3 is a schematic view of a sandwich assay embodiment of the quantitative lateral flow assay strip of FIG. 2 illustrating the condition of the strip upon partial migration of the of biological sample along the test strip body portion;

FIG. 4 is a schematic view of a sandwich assay embodiment of a quantitative lateral flow assay strip of FIG. 2 upon complete migration of the biological sample along the test strip body portion;

FIG. 5 is photographic image of eight sandwich assay quantitative lateral flow strips arranged side-by-side, which illustrate the proportional relationship between optical intensity and amylase concentration of the various samples tested.

FIG. 6 is a photographic image of a handheld point of care optical analyzer for receipt of the quantitative lateral flow test strips of the invention.

FIG. 7 is a schematic view of a competitive assay embodiment of a quantitative lateral flow assay strip of the invention illustrating the initial condition of the strip upon deposition of biological sample;

FIG. 8 is a schematic view of the competitive assay embodiment of a quantitative lateral flow assay strip of FIG. 7 illustrating the condition of the strip upon partial migration of the of biological sample along the test strip body;

FIG. 9 is a schematic view of a competitive assay embodiment of a quantitative lateral flow assay strip of FIG. 7 illustrating the condition of the strip upon complete migration of the of biological sample along the test strip body portion; and

FIG. 10 is photographic image of seven competitive assay quantitative lateral flow assay strips arranged side-by-side, which illustrate the inverse proportional relationship between optical intensity and estradiol concentration of the various samples tested.

FIG. 11A is an enlarged photographic image of a dropper bottle in accordance with the kit embodiment of the invention show in FIG. 12.

FIG. 11B is an enlarged plan view of a cartridge holding a test strip in accordance with another embodiment of the invention.

FIG. 11C is an enlarged photographic image of a pipette of the kit embodiment of the invention shown in FIG. 12

FIG. 12 is a plan view of a quantitative lateral flow assay kit in accordance with a still further embodiment of the invention.

### DETAILED DESCRIPTION OF THE PREFERRED EMBODIMENTS OF THE INVENTION

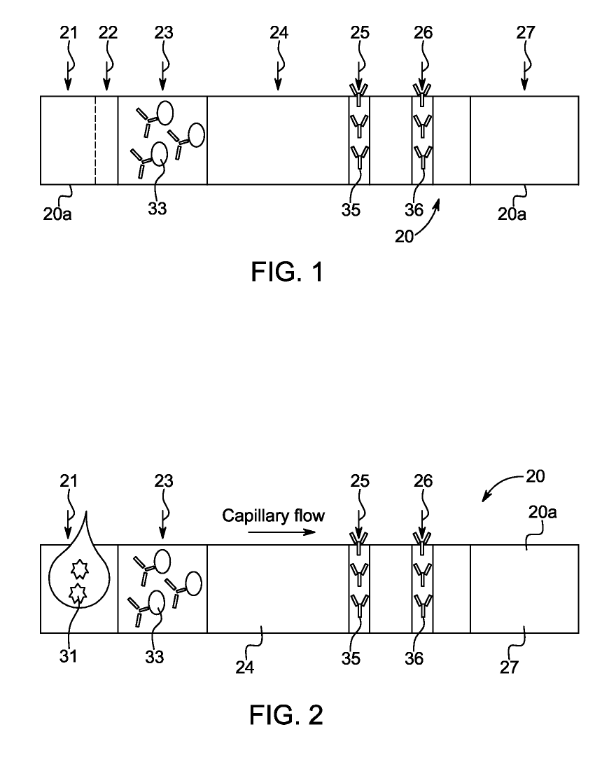

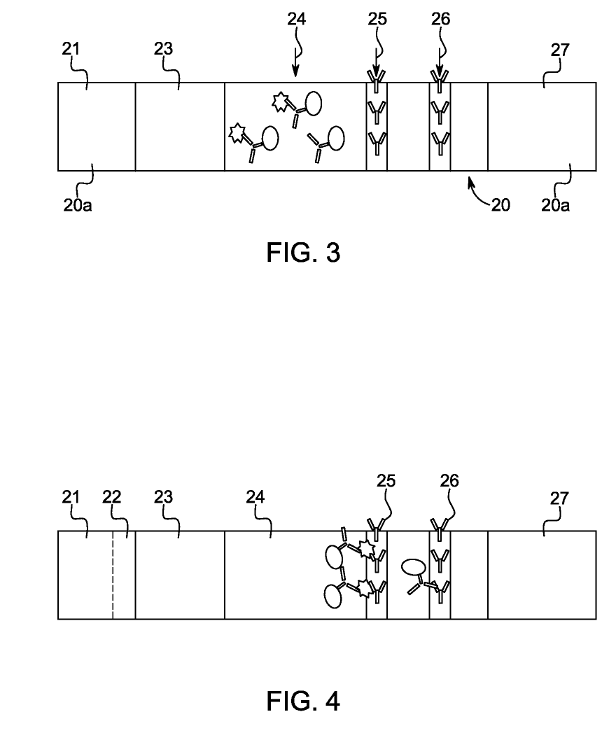

As illustrated in FIGS. 1-5, in one embodiment of the invention, a sandwich assay quantitative lateral flow assay strip 20 is provided, which includes, an elongated body portion 20a dimensioned for insertion into the strip receiver portion 52 of the point of care scanner 50 (best seen in FIG. 6); a sample pad 21 for deposit of a quantity of a biological sample containing an unknown quantity of the specific analyte 31; a conjugate pad 23 located adjacent to the sample pad and having an analyte/antibody conjugated to a gold particle 33, which are fixed to the conjugate pad 23 in a dry state; a capillary flow portion 24 adjacent to the conjugate pad 23 having a pore size selected to facilitate sample flow through the elongated body portion 20a; a test line portion 25 having an anti-analyte antibody 35 fixed in a dry state to the elongated body portion 20a of the strip 20; a control line 26 having anti-conjugate antibody 36 fixed in a dry state; and, an absorbent pad 27 for absorbing excess analyte or chase buffer applied to the body portion 20a for improving the flow rate through the capillary flow portion 24. Optionally, depending on the nature of the biological specimen from which the analyte is to be quantified, the strip 20 may include a barrier portion 22 to prevent cells and/or non-cellular particulates in the biological sample from entering the remainder of the strip and interfering with the optical density/intensity results. Preferably, the strip 20 is dimensioned for receipt in a cartridge 52, which provides additional structural stability to the strip 20. The cartridge 52 is preferably dimensioned for insertion into the strip receiver portion 52 of the point of care scanner 50.

### Salivary Amylase Sandwich Assay Test Strip Example

Turning in more detail to a more specific example, a sandwich assay strip in accordance with one embodiment of the invention is illustrated in FIGS. 1-5. Sandwich format quantitative lateral flow test strips for detecting salivary amylase protein were custom developed using goat anti-human salivary amylase polyclonal antibody [Pacific immunology Corp, CA, USA]. The antibody was raised using a highly purified human salivary amylase protein obtained from Abcam, USA as antigen. The process included the steps of obtaining a high flow rate nitrocellulose membrane from Millipore Corporation, Billerica, Mass., USA to form the capillary flow portions 24 of the strips 20b-20i and to underlie sample pad 21 (Schleicher & Schuell, 20 mm core, Grade 939 Paper), conjugate pad 23 (Millipore, 10 mm core), test line 25 (Glass Fiber), control line portion 26 and absorbent pad 27 (Schleicher & Schuell, 18 mm core, Grade 222 Paper). These were all assembled on an Adhesive Backing (G&L Precision Die Cutting, 3″ core, 0.010″ vinyl, PN#GL36106) to form a complete test strip. The custom developed goat anti-amylase antibody 35 was immobilized onto the test strip membranes 24 to form test lines 25 also using a reagent dispenser. The conjugate pad 23 was formed on the capillary flow portions 24. Polyclonal goat anti-human salivary amylase antibody that had been conjugated to 30 nm gold particles purchased from BIO Assay Works, Jamesville, Md., USA following the manufacturer's protocol was dried on the conjugate pad.

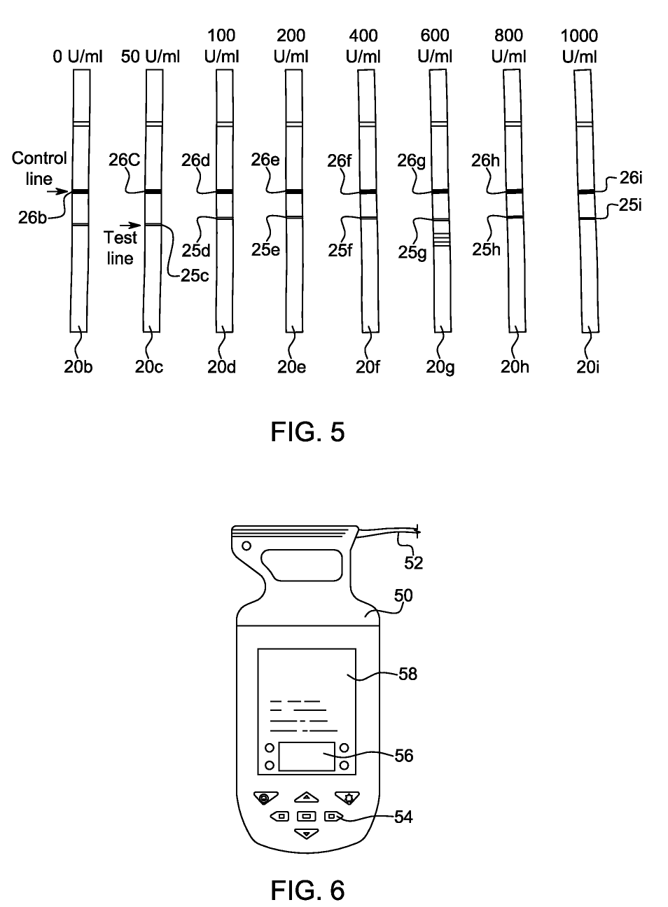

To prepare the amylase samples, purified human salivary amylase was obtained from Sigma Chemical and reconstituted in 1 mM Calcium Chloride to 1000 Units per ml. Eight different concentrations of amylase beginning at 50 Units per milliliter through 1000 Units per milliliter were prepared in Phosphate Buffered Saline (PBS). A sample size of 90 μls for each of the eight amylase solution concentrations shown in Table 1 and Graph 1, and used to test the strips for linear dose response at 1:750 in a Tris based Chase buffer (0.1M Tris, 0.15M NaCl, 2.5 mM EDTA, 0.5% Triton X-100, 0.1% Sodium azide, pH 10.00). The test was completed in 15 minutes. The strips were tested on three different days using amylase standards prepared on each of the respective days. A photographic image of the eight test strips 20b-20i arranged side-by-side from one of the three days is shown in FIG. 5, and it is evident from a simple visual inspection that the color intensity of the test lines 25g-25i of the strips of highest amylase concentrations were significantly darker in color than the test strips of the lowest concentrations 25c-25e. The color intensity of each of the test lines 25b-25i of the eight test strips 20b-020i were read using an RDS-1000 Scanner purchased from Detekt Biomedical, LLC of Austin Tex. Optionally, a point of care fluorescent scanner could be used if an Immunofluorescent stain having a fluorophore particle is substituted for the conjugated gold particles at the test line. The optical intensity results for each of the samples were plotted on the graph shown in Table 2 below and a linear regression analysis was performed on the intensity results, which yielded an r² value of 0.96. This linear regression score indicates that the sandwich assay quantitative lateral flow strips of the invention yield a strong linear dose response relationship whereby the test line displays an optical intensity which is directly proportional to the concentration (amount) of analyte in the sample.

TABLE 1
Comparison of the concentration of amylase and the mean Integral
Optical Density obtained from the RDS- 1000 scanner.

| Serial dilutions of | Integrated Optical Density |
|---|---|
| amylase standard (U/ml) | RDS- 1000 scanner |
| 0 | 1218 |
| 50 | 11712 |
| 100 | 24668 |
| 200 | 50413 |
| 400 | 60198 |
| 600 | 90689 |
| 800 | 101695 |
| 1000 | 118471 |

As can be seen from FIG. 5, the optical intensity of the control lines 26b-26i on each of the eight test strips 20b-20i show strong optical intensities, which are nearly a constant color. A visually strong control line 26b-26i result provides visual evidence that the test strip has been properly processed by the point of care test strip technician (typically a nurse or other similarly trained staff member). In this way, false negative results can be avoided since improper storage, handling or processing of the strip would cause the antibody 36 to fail to bind to the analyte so that the control line 26 would not register a visually intense signal. Further, false positive results can be avoided since the presence of a test line without a strong control line on a strip would indicate that an error in storage, handling, or processing of the strip had occurred.

Applicant has found that the quantitative lateral flow assay strips of the invention provide more consistent and reliable optical intensity readings after allowing the assay strips to dry prior to taking optical intensity measurements from their test lines. Drying of the quantitative lateral flow assay strips before optical intensity measurements are taken can be performed by gently heating the quantitative lateral flow assay strips, applying a desiccant (drying agent) to the qualitative lateral flow assay strips, or allowing the qualitative lateral flow assay strips to air dry over time. The accuracy and reliability of the quantitative lateral flow assay strips can be further improved by calibrating the scanner by comparing optical intensity measurements from a quality control quantitative lateral flow strip created for the specific type of analyte. The quality control quantitative lateral flow strip is created by adding a known quantity of the specific analyte within the therapeutically important expected range of quantities in a given type of biological sample to the sample pad of at least one quantitative lateral flow assay strip for the specific analyte. Then, a chase buffer is added to the sample pad and lateral flow of the analyte is allowed to occur. Thereafter, the newly created quality control quantitative lateral flow strip is dried. Next, the optical intensity at the test line of the at least one quality control strip is measured. Thereafter, the scanner can be calibrated by comparing the optical intensity of the known quantity of specific analyte from the quality control strip with optical intensities measured for unknown quantities of the specific analyte in the biological samples applied to the quantitative lateral flow assay strips.

In a point of care setting, the scanner 50 would need to be programmed to calculate analyte concentration from measured optical intensity for each of the quantitative lateral flow assay strips 20 for each specific analyte of interest to a particular point of care clinician. The linear relationship between analyte concentration and test strip optical intensity for sandwich assay type strips of the invention would be calculated via standardization procedures similar to those described above for the salivary amylase test strips 20b-20i. In a similar way, a formula for deriving analyte concentration from optical density readings for each different analyte specific test strip would be determined and programmed into the scanner 50 for each of the appropriate analytes of interest to a particular clinician or medical practice. Applicant has found that the Detekt RDS-1000 is a suitable point of care optical analyzer to provide accurate quantification of test strips of the invention. The RDS-1000 reader includes a universal cartridge 52, which is shaped to secure a variety of qualitative lateral flow test strips in order to accurately optically analyze defined portions of those strips upon insertion of the universal cartridge 52 into the analyzer 50. The test strips 20 of the invention are preferably shaped and sized to securely fit in the Detekt universal cartridge 52, but may also be shaped and sized to securely fit into a variety of other commercially available point of care optical analyzers, such as, ROSA-pearl X reader, CHARM Sciences, USA; Diagnostic Consulting Network, Carlsbad, Calif., USA; and Bio Assay works, Jamesville, Md., USA. Optionally, the strips may be loaded into their own customized cassette for analysis by either a customized optical reader made specifically to accommodate such a cassette or by the other commercially available point of care optical readers.

The Detekt scanner 50 is highly programmable device as it is designed to be used with a wide variety of commercially available qualitative lateral flow test strips. These qualitative strips are not designed to provide linear dose response optical intensities in response to varying concentrations of an analyte. Accordingly, in one preferred method of the invention, the Detekt scanner 50 would need to be re-programmed to interpret the optical intensity readings for quantitative lateral flow test strips as definite concentrations. It would be optimal if the scanner 50 was programmed for each of different type of quantitative test strips of the invention that would likely to be use in a particular clinic, hospital department, or specialized medical practice. After such re-programming, the scanner 50 would be capable of calculating a concentration (amount) of the analyte specific to the specific type of quantitative strips loaded into the universal cartridge 52 in response to operator input through a key pad 54 or touch pad 56 to access the appropriate analyte specific program. After activation and selection of the appropriate program, the scanner 50 would show the calculated concentration value to an operator in real time on a display 58 on the scanner 50. Optionally, the scanner 50 could be linked, either wirelessly or via a UBS connection, to the point of care facilities computer system so that the rest results were stored electronically in the memory of that system in a specified patient's medical records.

In the salivary amylase example described herein, the test strips 20b-20i proved capable of quantifying very small sample sizes on the order of 70μ liters. This shows that the strips are highly sensitive so that very small volume samples can yield good quantitative results. This sensitivity and low cost provides the clinician with the option of splitting biological samples into A and B portions so that a second test can be performed by a different technician using a fresh strip and different optical analyzer on the same sample if an unusual or unexpected test result is obtained in the A portion assay. In this way, the unusual or unexpected result can be confirmed or rejected by careful assaying of the B portion of the sample. Further, as will be illustrated below, in the competitive assay analysis described in the second example provided herein, the sensitivity of the strip allows for sufficiently small samples that pin pick (or finger prick) methods utilizing a single drop of blood can be used to quantify a blood serum analyte. This can reduce or eliminate the need for a trained phlebotomist to draw large samples of blood via a traditional venipuncture methodology. However, certain analytes may still require the drawing larger blood samples so that more traditional venipuncture methods may still sometimes be necessary for certain types of quantitative test strips of the invention. Moreover, in some cases, the sensitivity of the strips of the invention will be sufficient to utilize saliva as the sample from which analyte concentration is determined so that even a pin prick blood sample will not be necessary to provide accurate quantification of the analyte.

The applicant has also had success preparing sandwich assay quantitative lateral flow assay strips specific for β-human chorionic gonadotropin, which are similar in most respects to the sandwich assay quantitative lateral flow assay strip for amylase described immediately above. The β-human chorionic gonadotropin quantitative lateral assay strips can be used to provide a pregnancy test, which incorporates a quantitative component that can prove useful for obstetricians and gynecologists in treating new patients.

Moreover, the applicant has also had success preparing sandwich type quantitative lateral flow assay strips for first trimester screening of pregnant women. Separate quantitative sandwich assay strips have been created to quantify both Pregnancy-associated plasma protein-A (PAPP-A) and free β-human chorionic gonadotropin. Quantitative analysis for either of those analytes is useful to obstetricians and gynecologists for treating and advising pregnant patients during their first trimester of pregnancy.

Furthermore, the applicant is also had success preparing sandwich type, quantitative lateral flow assay strips for use in the selection of the most viable embryos from a group of fertilized embryos created during artificial fertilization procedures. In this process, quantitative lateral flow assay strips are created, which can be used to quantify total hCG levels in a cohort of developing embryos. Based on the quantification using the quantitative lateral flow assay strips specific for total hCG in each embryo of the cohort, one or more of the most viable embryos can be selected for implantation into the patient.

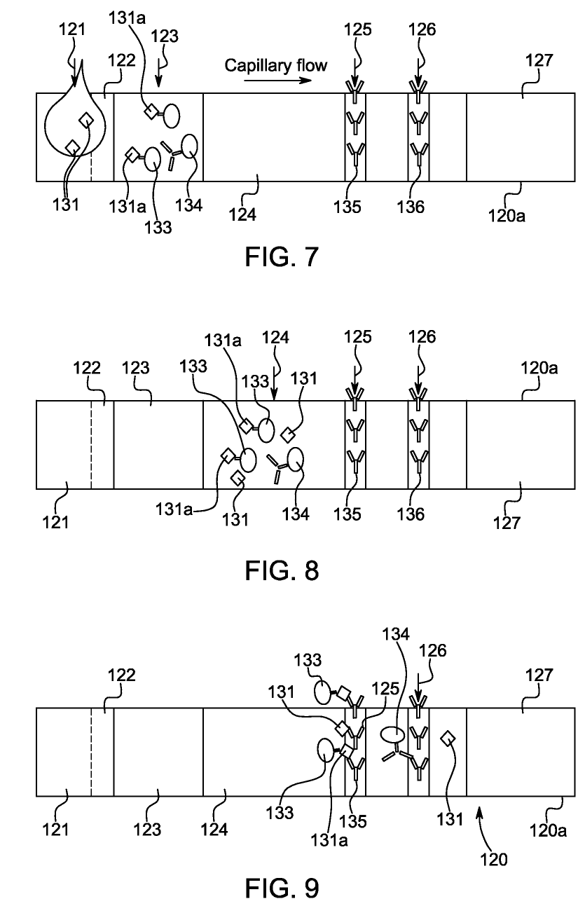

Another embodiment of the quantitative lateral flow assay strips of the invention is illustrated FIGS. 7-10. This embodiment of the invention includes a competitive assay quantitative lateral flow assay strip 120 having an elongated body portion 120a dimensioned for insertion into the strip receiver portion 52 of the point of care scanner 50 (best seen in FIG. 6); a sample pad 121 for deposit of a quantity of a biological sample containing an unknown quantity of the specific analyte 131; a conjugate pad 123 located adjacent to the sample pad and having an anti-analyte/antibody conjugated to a gold particle 133 as well as an analyte bound anti-analyte antibody conjugated to a gold particle 134, which are both fixed to the conjugate pad 123 in a dry state; a capillary flow portion 124 adjacent to the conjugate pad 123 having a pore size selected to facilitate sample flow through the elongated body portion 120a; a test line portion 125 having an anti-analyte antibody 135 fixed in a dry state to the elongated body portion 120a of the strip 120; a control line 126 having anti-conjugate antibody 136 fixed in a dry state; and, an absorbent pad 127 for absorbing excess analyte or chase buffer applied to the body portion 120a for improving the flow rate through the capillary flow portion 124. Optionally, depending on the nature of the biological specimen from which the analyte is to be quantified, the strip 120 may include a barrier portion 122 to prevent cells and/or non-cellular particulates in the biological sample from entering the remainder of the strip and interfering with the optical density results. Preferably, the strip 120 is dimensioned for receipt in a cartridge 52. The cartridge 52 is preferably dimensioned for insertion into the strip receiver portion 52 of the point of care scanner 50.

### Estradiol Competitive Assay Test Strip Example

Turning in more detail to the competitive assay strip in accordance with another embodiment of the invention is illustrated in FIGS. 7-10. The competitive quantitative lateral flow test strip 120 for detecting estradiol utilizes a custom developed rabbit anti-estradiol antibody (Clinical Endocrinology Laboratory, University of California, Davis, Calif. 95616). The antibody was raised using a highly purified human estradiol obtained from Abcam, USA as antigen. The process included the steps of obtaining a high flow rate nitrocellulose membrane from Millipore Corporation, Billerica, Mass., USA to form the capillary flow portions 124 of the strips 120b-120h and to underlie sample pad 121 (Schleicher & Schuell, 20 mm core, Grade 939 Paper), conjugate pad 123 (Millipore, 10 mm core), test line 125 (Glass Fiber), control line portion 126 and absorbent pad 127 (Schleicher & Schuell, 18 mm core, Grade 222 Paper). These were all assembled on an Adhesive Backing (G&L Precision Die Cutting, 3″ core, 0.010″ vinyl, PN#GL36106) to form a complete test strip. The custom developed rabbit anti-estradiol antibody 135 was immobilized onto the test strip membranes 124 to form test lines 125 also using a reagent dispenser. The conjugate pad 123 was formed on the capillary flow portions 124. BSA-Oxime-estradiol from Sigma-Aldrich, St. Louis, Mo., USA (or similar estradiol complexes such as estradiol 3-CME-BSA, estradiol 6-CMO-BSA, estradiol 17-Hemisuccinate-BSA, Fitzgerald Industries International, Acton, Mass. 01720; estradiol 17B-BSA, Calbioreagents, San Mateo, Calif. 94403) that had been conjugated to 30 nm gold particles purchased from BIO Assay Works, Jamesville, Md., USA following the manufacturer's protocol was dried on the conjugate pad.

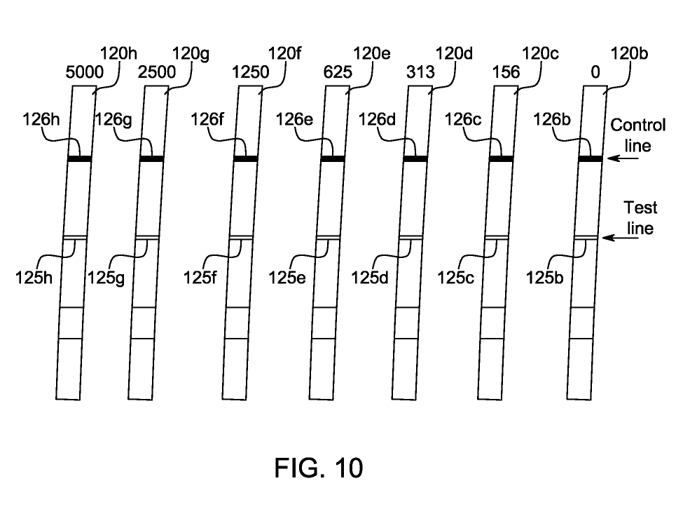

To prepare the estradiol samples, purified human estradiol was obtained from Sigma-Aldrich, St Louis, Mo., USA and diluted in Phosphate Buffered Saline (PBS) containing 0.1% BSA to yield seven different concentrations of estradiol beginning at 156 picograms per milliliter through 5000 picograms per milliliter. A sample size of 50 μls for each of the seven estradiol solution concentrations shown in Table (2) and Graph 2, and used to test the strips for linear dose response using Tris based Chase buffer (0.1M Tris, 0.15M NaCl, 2.5 mM EDTA, 0.5% Triton X-100, 1 mM 8-Anilino-1-naphthalenesulfonic acid, 0.1% Sodium azide, pH 10.00). The test was completed in 15 minutes. The strips were tested on three different days using estradiol standards prepared on each of the respective days. A photographic image of the seven test strips 120b-120h arranged side-by-side from one of the three days is shown in FIG. 10, and it is evident from a simple visual inspection that the color intensity of the test lines 125g-125h of the strips of highest estradiol concentrations were significantly lighter in color than the test strips of the lowest concentrations 25c-25e. The color intensity of each of the test lines 125b-125h of the seven test strips 120b-120i were read using an RDS-1000 Scanner purchased from Detekt Biomedical, LLC of Austin Tex.. The optical intensity results for each of the samples were plotted on the graph and a linear regression analysis was performed on the intensity results, which yielded an r² value of 0.96. This linear regression score indicates that the competitive assay quantitative lateral flow strips of the invention yield a strong linear dose response relationship whereby the test line displays an optical intensity which is inversely proportional to the concentration (amount) of analyte in the sample.

TABLE 2
Comparison of the concentration of estradiol and the mean Integral
Optical Density obtained from the RDS - 1000 scanner.

| Serial dilutions of | Integrated Optical Density |
|---|---|
| estradiol standard (pg/ml) | RDS- 1000 scanner |
| 5000 | 4.37E+06 |
| 2500 | 5.29E+06 |
| 1250 | 5.60E+06 |
| 625 | 6.36E+06 |
| 312 | 6.70E+06 |
| 156 | 6.84E+06 |
| 0 | 7.41E+06 |

As can be seen from FIG. 10, the optical intensity of the control lines 126b-126h on each of the seven test strips 120b-120h show strong optical intensities, which are nearly a constant color. A visually strong control line 126b-126h result provides visual evidence that the test strip has been properly processed by the point of care test strip technician (typically a nurse or other similarly trained staff member). In this way, false negative results can be avoided since improper storage, handling or processing of the strip would cause the antibody 136 to fail to bind to the analyte so that the control line 126 would not register a visually intense signal. Further, false positive results can be avoided since the presence of a test line without a strong control line on a strip would indicate that an error in storage, handling, or processing of the strip had occurred.

In a point of care setting, the scanner 50 would need to be programmed to calculate analyte concentration from measured optical intensity for each of the competitive quantitative lateral flow assay strips for each specific analyte of interest to a particular point of care clinician. The inversely proportional linear relationship between analyte concentration and test strip optical intensity for competitive assay type strips of the invention would be calculated via standardization procedures similar to those described above for the estradiol test strips 120b-120h. In a similar way, a formula for deriving analyte concentration from optical density readings for each different analyte specific test strip would be determined and programmed into the scanner 50 for each of the appropriate analytes of interest to a particular clinician or medical practice. Applicant has found that the Detekt RDS-1000 is a suitable point of care optical analyzer to provide accurate quantification of test strips of the invention. The RDS-1000 reader includes a universal cartridge 52, which is shaped to secure a variety of qualitative lateral flow test strips in order to accurately optically analyze defined portions of those strips upon insertion of the universal cartridge 52 into the analyzer 50. The test strips 120 of the invention are preferably shaped and sized to securely fit in the Detekt universal cartridge 52, but may also be shaped and sized to securely fit into a variety of other commercially available point of care optical analyzers, such as, ROSA-pearl X reader, CHARM Sciences, USA; Diagnostic Consulting Network, Carlsbad, Calif., USA; and Bio Assay works, Jamesville, Md., USA. Optionally, the strips may be loaded into their own customized cassette for analysis by either a customized optical reader made specifically to accommodate such a cassette or by the other commercially available point of care optical readers.

The Detekt scanner 50 is highly programmable device as it is designed to be used with a wide variety of commercially available qualitative lateral flow test strips. These qualitative strips are not designed to provide linear dose response optical intensities in response to varying concentrations of an analyte. Accordingly, in one preferred method of the invention, the Detekt scanner 50 would need to be re-programmed to interpret the optical intensity readings for quantitative lateral flow test strips as definite concentrations. It would be optimal if the scanner 50 was programmed for each of different type of quantitative test strip of the invention that would likely to be use in a particular clinic, hospital department, or specialized medical practice. After such re-programming, the scanner 50 would be capable of calculating a concentration (amount) of the analyte specific to the specific type of quantitative strips loaded into the universal cartridge 52 in response to operator input through a key pad 54 or touch pad 56 to access the appropriate analyte specific program. After activation and selection of the appropriate program, the scanner 50 would show the calculated concentration value to an operator in real time on a display 58 on the scanner 50. Optionally, the scanner 50 could be linked, either wirelessly or via a UBS connection, to the point of care facilities computer system so that the rest results were stored electronically in the memory of that system in a specified patient's medical records. In the estradiol example described herein, the test strips 120b-120h proved capable of quantifying very small sample sizes on the order of 50μ liters. This shows that the strips are highly sensitive so that very small volume samples can yield good quantitative results. This sensitivity and low cost provides the clinician with the option of splitting biological samples into A and B portions so that a second test can be performed by a different technician using a fresh strip and different optical analyzer on the same sample if an unusual or unexpected test result is obtained in the A portion assay. In this way, the unusual or unexpected result can be confirmed or rejected by careful assaying of the B portion of the sample. Further, in the competitive assay analysis described in the second example provided herein, the sensitivity of the strip allows for sufficiently small samples that pin prick (or finger prick) methods utilizing a single drop of blood can be used to quantify a blood serum analyte. This can reduce or eliminate the need for a trained phlebotomist to draw large samples of blood via a traditional venipuncture methodology. However, certain analytes may still require the drawing larger blood samples so that more traditional venipuncture methods may still sometimes be necessary for certain types of quantitative test strips of the invention. Moreover, in some cases such as with estradiol saliva levels of IVEFT patients, the sensitivity of the strips of the invention will be sufficient to utilize saliva as the sample from which analyte concentration is determined so that even a pin prick will not be necessary to provide accurate quantification of the analyte.

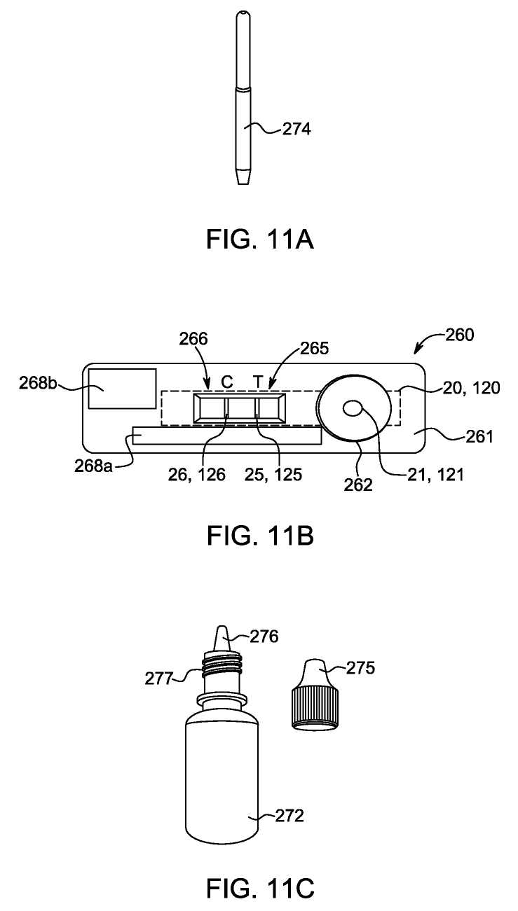

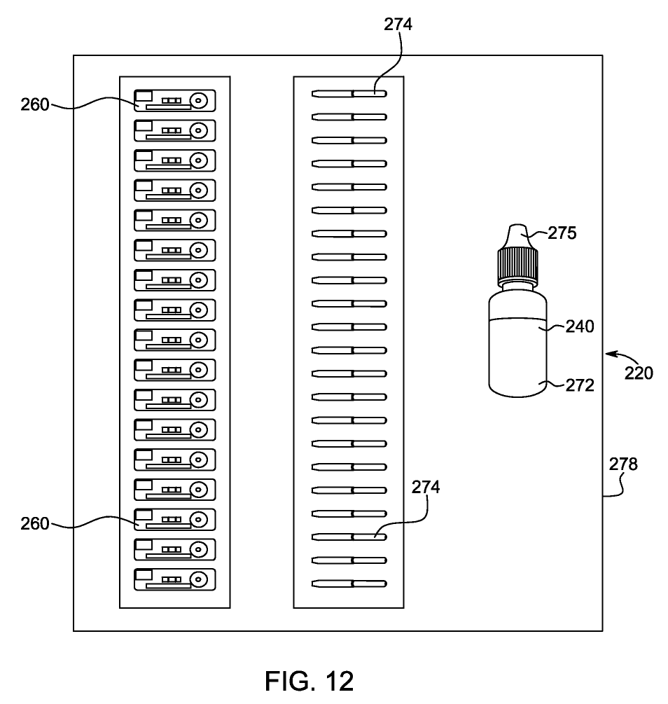

In another aspect of the invention, a quantitative lateral flow assay kit 220 is provided, which contains the consumables constituents necessary for quantitative analysis of a specific analyte in a point of care operated scanner 50. One embodiment of the kit of the invention is shown in FIGS. 11A-C and 12. Depending upon the analyte of interest and the level of sensitivity necessary for the assay, the kit 220 may include sandwich assay strips (20), competitive assay strips (120) or both types of strips (20, 120). Preferably, the kit includes at least the following: (1) a container of chase buffer 240 selected to optimize sample lateral flow through the test strip and to chemically stabilize a specific analyte during the assay procedure and (2) a plurality of quantitative linear analyte concentration assay strips having (a) an elongated body portion (20a, 120a) dimensioned for insertion into a strip receiver portion 52)of the point of care scanner 50; (b) a sample pad (21, 121) for deposit of a known quantity of a biological sample containing an unknown quantity of the specific analyte (31, 131); (c) a conjugate pad (23, 123) located adjacent to the sample pad (21,121) and having an anti-analyte antibody conjugated to an optically detectable particle, which are fixed to the conjugate pad in a dry state; (d) a capillary flow portion adjacent (24, 124) to the conjugate pad (23,123) having a pore size selected to facilitate sample flow through the elongated body portion; (e) a test line portion (25,125) having an anti-analyte antibody fixed in a dry state to the body portion of the strip, the test line (25,125) displaying variable optical intensity in linear proportion to the concentration (amount) of analyte in the sample; and (f) a control line (26, 126) having anti-conjugate antibody fixed in a dry state, the control line displaying a relatively stable optical intensity in the presence of the analyte and little to no optical intensity in the absence of the analyte. In the competitive assay technique strips, the optical intensity is inversely proportional to analyte concentration. For the sandwich assay technique strips the optical intensity is directly proportional to analyte concentration.

The applicant believes that other steroid class hormones, such as progesterone, are good candidates for creation of quantitative lateral flow assay strips of the competitive assay type described above with respect to Estradiol. Estradiol is, of course, a steroid class hormone that chemically behaves in a relatively similar way to progesterone so applicant believes preparation of a quantitative lateral flow assay strip specific to progesterone will follow an equivalent protocol to the estradiol competitive assay strip protocol described herein. Quick, accurate quantification of progesterone levels would be useful in human fertility treatments and to animal breeding programs to determine when an animal is entering an ovulation cycle (coning into “heat”).

In the embodiment of the kit shown in FIGS. 11A-C and 12, each of the plurality of quantitative linear analyte concentration assay strips (20, 120) is contained within a custom designed cassette 260. Each cassette 260 includes a housing portion 261 which encloses much of the elongated strip body (20a, 120a). The housing 261 is preferably injection molded from a chemically stable thermoplastic resin in two halves that are then snap fit together around the test strip (20,120). The housing portion 261 includes an analyte input window 262, which lines up with the sample pad portion (21, 121) of the strips (20, 120). The housing portion 261 further includes a test line window area 265, which is aligned with test lines (25, 125) of strips (20,120) so that the test lines (25,125) of the strips (20, 120) are visible from the exterior of the cartridge 260. Similarly, the housing portion 261 includes a control line window area 266, which is aligned with control lines (26, 126) of strips (20,120) so that the control lines (26,126) of the strips (20, 120) are visible from the exterior of the cartridge 260. The use of the custom cartridge 260 is preferred for the kit 220 since it provides individual protection for the delicate strips during shipping, storage, processing, and analysis by the point of care operated scanner 50. The cassette 260 may also be provided with identification portions 268a and 268b upon which patient identification labels can be affixed, patient data can be written, and analyte information can be recorded. If low-cost, high volume test strips are necessary for a particular application, it may preferable to omit the cartridge and provide the end user with a continuous roll of strips that are joined end to end with perforation defining the boundaries of each strip or to provide a strip dispenser having a housing which holds a large number of test strips that may be serially removed as needed.

The preferred kit 220 designed to accommodate relatively high volume testing of samples of a particular analyte by the end user, typically a technician at a point of care facility. Accordingly, the kit 220 will include a bulk number of individually sealed cassettes 260 (between about 10-50 units) containing fully assembled strips (20, 120) specific to a single analyte. The preferred kits will further include a 1-5 ml plastic dropper bottle 272 that dispenses a fixed volume of drop of the chase buffer 240 at a time, (Zinsser NA, Northridge, Calif., USA) and contain a sufficient volume of chase buffer 240 to assay more than the bulk number of strips in a kit. The plastic dropper includes an internally threaded screw cap 275 which engages external threads 277 located near the dropper tip 276. It will also include a matching bulk number (between about 10-50) of disposable one time use 5-50 μl plastic pipettes 274 (“MicroSafe” Tube: Safetec, Ivyland, Pa., USA) for handling the biological samples to be assayed by the strips of the kit. The kit is housed in a suitable container 278 to protect the sealed cassettes 260, dropper bottle 272, and pipettes 274.

What is claimed:

1. A quantitative lateral flow assay strip to provide quantitative analysis of a specific analyte using a point of care scanner having an assay strip receiver portion, the quantitative lateral flow assay strip comprising:
a body portion dimension for insertion into the strip receiver portion of the point of care scanner;
a sample pad for deposit of a known quantity of a biological sample containing an unknown quantity of the specific analyte;
a conjugate pad located adjacent to the sample pad having an analyte/antibody conjugated to an optically detectable particle, the analyte/antibody optically detectable particle conjugate fixed to the conjugate pad in a dry state;
a capillary flow portion adjacent to the conjugate pad having a pore size selected to facilitate sample flow through the body portion;
a test line portion having an anti-analyte antibody fixed in a dry state to the body portion of the strip, the test line displaying an optical intensity which is proportional to the quantity of the specific analyte within an expected therapeutically important range of specific analyte quantities for the biological sample so that the unknown amount of specific analyte can be accurately calculated by analyzing the optical intensity of the test line; and
a control line having anti-conjugate antibody fixed in a dry state, the control strip displaying a relatively constant optical intensity in the presence of the specific analyte to ensure that the assay strip has been appropriately processed.

2. The quantitative lateral flow assay strip of claim 1 wherein the assay strip is calibrated to provide quantifiable optical intensity readings in the presence of the expected range of sample specific analyte quantities, the amount of anti-analyte antibody fixed to the test line being selected to provide a predetermined range of optical intensities that fall within the range of detectable and quantifiable optical intensities of a point of care scanner and wherein the unknown amount of specific analyte in the sample can be calculated by pre-programming the point of care scanner to calculate the unknown amount of specific analyte in the sample from the optical intensity reading detected for an assay strip within the predetermined range of optical intensities.

3. The quantitative lateral flow assay strip of claim 1 wherein the assay strip is dimensioned for receipt in a cartridge housing having predetermined dimensions, the predetermined dimensions of the cartridge housing being selected to provide an operable interface with assay strip receiving portion of the point of care scanner to align the test line portion of the assay strip with an optical intensity reading portion of the point of care scanner.

4. The quantitative lateral flow assay strip of claim 1 wherein the assay strip is enclosed within a cassette having a housing portion injection molded from a chemically stable thermoplastic resin, the cassette having two housing halves which are snap fit together to substantially enclose the assay strip.

5. The quantitative lateral flow assay strip of claim 1 wherein the housing portion further includes an analyte input window defined by an input frame which extends around the periphery of the sample pad portion of the strip, the analyte input window providing an opening for deposit of the biological sample on the sample pad portion.

6. The quantitative lateral flow assay strip of claim 1 wherein the housing portion further includes a test line window area defined by a test line frame which extends around the periphery of the test line portion of the assay strip, the test line window area providing access to the test line for the point of care scanner to read the optical intensity of test line portion of the assay strip.

7. The quantitative lateral flow assay strip of claim 1 wherein the housing portion further includes a control line window area, defined by a control line frame which extends around the periphery of the control line portion of the assay strip, the control line window area being transparent so that the control line of the assay strip is visible from the exterior of the cartridge.

8. The quantitative lateral flow assay strip of claim 1 wherein the housing portion further includes a substantial planar identification portion having an information display surface upon which patient identification or analyte information can be recorded.

9. The quantitative lateral flow assay strip of claim 1 wherein a known quantity of the specific analyte is fixed in a dry state to the capillary flow portion of the assay strip body and wherein the unknown quantity of the specific analyte in the biological sample is inversely proportioned to the optical intensity displayed at the test line.

10. A quantitative lateral flow assay kit containing consumables constituents necessary for quantitative analysis of a specific analyte using a point of care operated scanner, the kit comprising: (1) a chase buffer selected to optimize sample lateral flow through the test strip and to chemically stabilize the specific analyte during the assay procedure and (2) a quantitative lateral flow assay strip having, (a) a body portion dimensioned for operable interface with the point of care scanner; (b) a sample pad for deposit of a known quantity of a sample containing an unknown quantity of the specific analyte; (c) a conjugate pad located adjacent to the sample pad and having an analyte/antibody conjugated to an optically detectable particle, the analyte/antibody optically detectable particle conjugate being fixed to the conjugate pad in a dry state; (d) a capillary flow portion adjacent to the conjugate pad having a pore size selected to facilitate sample flow through the body portion; (e) a test line portion having an anti-analyte antibody fixed in a dry state to the body portion of the strip, the test line displaying variable optical intensity readings proportional to the quantity of specific analyte in the sample; and (f) a control line having anti-conjugate antibody fixed in a dry state, the control line displaying a relatively stable optical intensity in the presence of the analyte.

11. The quantitative lateral flow assay kit of claim 10 wherein the quantitative lateral flow assay strip and scanner are calibrated by analyzing a range of known variable optical intensities which correlate with a clinically meaningful expected range of specific analyte concentrations in a specific type of biological sample, wherein the correlation between optical intensities and the range of expected specific analyte concentrations are preprogrammed into the memory of the optical intensity reader for the specific assay strip, and wherein the scanner is preprogramed to calculate the specific analyte concentration and to display the calculated concentration value to an operator in real time at the point of care facility.

12. The quantitative lateral flow assay kit of claim 10 wherein the quantitative lateral flow assay strip further includes a barrier portion located between the sample pad and the conjugate pad to prevent cells and non-cellular particulates that may be found in the biological sample from entering the portion of the assay strip downstream from the sample pad.

13. The quantitative lateral flow assay kit of claim 10 wherein the quantitative lateral flow assay strip is of a sandwich assay design, and the optical intensity of the assay strip test line is directly proportional to specific analyte concentration

14. The quantitative lateral flow assay kit of claim 10 wherein the quantitative lateral flow assay strip is of a competitive assay design, such that the optical intensity of the assay strip test line is inversely proportional to specific analyte concentration.

15. A method of quantifying a therapeutically important specific analyte in a biological sample utilizing a quantitative lateral flow assay strip and a point of care optical scanning device, the method comprising the steps of:
(a) providing a quantitative lateral flow assay strip having, (i) a body portion dimensioned for operable interface with a point of care scanner; (ii) a sample pad for deposit of a known quantity of a biological sample containing an unknown quantity of the specific analyte; (iii) a conjugate pad located adjacent to the sample pad and having an analyte/antibody conjugated to an optically detectable particle, the analyte/antibody optically detectable particle conjugate being fixed to the conjugate pad in a dry state; (iv) a capillary flow portion adjacent to the conjugate pad having a pore size selected to facilitate sample flow through the body portion; and (v) a test line portion having an anti-analyte antibody fixed in a dry state to the body portion of the strip, the test line displaying variable optical intensity proportional to the quantity of specific analyte in the sample;
(b) providing a point of care scanner calibrated and programmed to quantify the amount of specific analyte in the biological sample;
(c) applying the biological sample having an unknown quantity of specific analyte to the sample pad of the quantitative lateral flow assay strip;
(d) applying a chase buffer selected to facilitate lateral sample flow through the quantitative lateral flow assay strip and to chemically stabilize the specific analyte during the assay procedure;
(e) acquiring an optical intensity reading from the test line of the quantitative lateral flow assay strip, and
(f) calculating the quantity of the specific analyte in the biological specimen from the optical intensity reading of the test line of the quantitative lateral flow assay strip.

16. The method of quantifying a therapeutically important specific analyte in a biological sample of claim 15 further including the further steps of (a) calibrating the quantitative lateral flow assay strip and scanner by analyzing a range of known variable optical intensities which correlate with a clinically meaningful expected range of specific analyte concentrations in a specific type of biological sample, (b) preprogramming the correlation between optical intensities and the range of expected specific analyte concentrations are preprogrammed into the memory of the optical intensity reader for the specific assay strip, (c) preprogramming the scanner to calculate the specific analyte concentration and to display the calculated concentration value to an operator in real time at the point of care facility.

17. The method of quantifying a therapeutically important specific analyte in a biological sample of claim 15 further including the step of providing a control line having an anti-conjugate antibody fixed in a dry state, the control line displaying a relatively constant optical intensity in the presence of the conjugate after processing the quantitative lateral flow assay strip to ensure correct processing of each of the quantitative lateral flow assay strips.

18. The method of quantifying a therapeutically important specific analyte in a biological sample of claim 15 further including the step of drying the quantitative lateral flow assay strip after applying the chase buffer and before acquiring the optical intensity reading from the test line.

19. The method of quantifying a therapeutically important specific analyte in a biological sample of claim 15 further including the steps of (a) creating at least one quality control strip having a known quantity of the specific analyte within the therapeutically important expected range of quantities in a given type of biological sample by adding a known quantity of the specific analyte to at least one quantitative lateral flow assay strip for the specific analyte; (b) reading the optical intensity at the test line of the at least one quality control strip; and (c) calibrating the scanner based on the at least one quality control strip optical intensity reading.

20. A method of manufacturing a quantitative lateral flow assay strip for quantifying the amount of a therapeutically important specific analyte in a biological sample for use with at least one specific type of scanner, the method comprising the steps of:
(a) selecting a specific analyte having a therapeutically important expected range of quantities in a given type of biological sample;
(b) determining the amount of anti-analyte antibody to fix to a test line portion of quantitative lateral flow assay strip to provide a range of optical intensities in response to the application of the biological specimen that fall within the range of detectable and quantifiable optical intensities for the at least one specific scanner;
(c) selecting a capillary flow stock material that permits capillary, lateral flow of the specific analyte;
(d) shaping a body portion of the assay strip from the selected capillary flow stock material;
(e) mounting a sample pad material onto the body portion of the assay strip for deposit of a known quantity of a biological sample containing an unknown quantity of the specific analyte;
(f) mounting a conjugate pad adjacent to the sample pad;
(g) creating a conjugate pad by fixing an analyte/antibody conjugated to an optically detectable particle in a dry state to the body portion downstream from the sample pad;
(h) constructing a test line portion by fixing in a dry state the determined amount of anti-analyte antibody to the body portion of the strip downstream from the conjugate pad, the test line providing an optical intensity proportional to the amount of analyte in the biological sample within the range of detectable and quantifiable optical intensities for the at least one scanner; and
(i) constructing a control line having anti-conjugate antibody fixed in a dry state, the control line displaying a relatively stable optical intensity in the presence of the analyte.

21. The method of manufacturing a quantitative lateral flow assay strip of claim 18 including the further step of adding a known quantity of specific analyte within the therapeutically important expected range of quantities in a given type of biological sample to the sample pad of at least one quantitative lateral flow assay strip and a chase buffer to the sample pad to create a lateral flow quality control strip; the lateral flow quality control strip providing an optical intensity reading when read by the scanner for comparison with the optical intensity readings of quantitative lateral flow assay strips with biological samples having unknown quantities of the specific analyte applied thereto.

22. The method of manufacturing a quantitative lateral flow assay strip of claim 18 including the further step of fixing a known quantity of the specific analyte in a dry state to the capillary flow portion of the assay strip body between the sample pad and the test line and wherein the unknown quantity of the specific analyte in the sample is inversely proportioned to the optical intensity displayed at the test line.
